(19) United States
(12) Patent Application Publication
(10) Pub. No.: US 2023/0257259 A1
(43) Pub. Date: Aug. 17, 2023

(54) MICROROBOT AND MANUFACTURING METHOD THEREOF

(71) Applicant: NATIONAL CHENG KUNG UNIVERSITY, Tainan (TW)

(72) Inventors: Chia-yuan Chen, Tainan (TW); Chun-fang Wang, Tainan (TW)

(73) Assignee: NATIONAL CHENG KUNG UNIVERSITY, Tainan (TW)

(21) Appl. No.: 17/806,273

(22) Filed: Jun. 10, 2022

(30) Foreign Application Priority Data
Feb. 15, 2022 (TW) 111105501

(51) Int. Cl. B82B 3/00; B25J 7/00

(52) U.S. Cl. CPC B82B 3/0033; B82B 3/0047; B25J 7/00

(57) ABSTRACT

A microrobot and manufacturing method thereof are provided. The microrobot includes a first block, a second block, and a third block connected with each other. The first block is disposed between the second block and the third block. The first block includes polydimethylsiloxane. The second block and the third block include a mixture, and the mixture includes polydimethylsiloxane and neodymium magnet particles. The manufacturing method of the microrobot includes the steps of providing a first acrylic mold with an accommodating space and a second acrylic mold with a U-shaped groove; injecting polydimethylsiloxane into the accommodating space; placing the second acrylic mold in the accommodating space; taking out the second acrylic mold and injecting the mixture into the accommodating space to obtain a microrobot. Placing the microrobot on an electromagnet platform can achieve an object of mixing and dissolving an embolism in a flow channel.

### CROSS REFERENCE TO RELATED APPLICATIONS

This application claims the priority of Taiwan Patent Application No. 111105501, filed on Feb. 15, 2022, titled “MICROROBOT AND MANUFACTURING METHOD THEREOF”, and the disclosure of which is incorporated herein by reference.

### FIELD OF INVENTION

The present disclosure relates to the technical field of microrobot, and particularly, to the microrobot for cleaning thrombus. The present disclosure also relates to a manufacturing method of the microrobot.

### BACKGROUND OF INVENTION

In the 20th century, with the maturity of microcontrollers, microscale manufacturing technology, and microelectromechanical technology, microrobots with microscale dimensions have been developed to reduce the manufacturing cost of mechanisms and components. In the 21st century, the microrobots are widely used in biomedical fields such as drug delivery and cardiovascular disease treatment.

Cardiovascular disease is one of the main causes of human death and disability. For the treatment of cardiovascular disease, studies have shown that treatment within three hours of cerebral stroke can not only increase the survival rate of patients, but also reduce disability and increase the chance of recovery. At present, the methods to shorten the time required for cerebral stroke treatment comprise the improvement of anticoagulant drugs and the use of mechanical thrombectomy devices. However, the improvement of anticoagulant drugs requires high research and development costs, and it is necessary to consider the possible side effects and harm to the human body during the research and development process of the anticoagulant drugs. Moreover, for the mechanical thrombectomy device, it is also necessary to consider the possible harm to the human body during the research and development process of the mechanical thrombectomy device, and its manufacturing cost is high.

By contrast, the manufacturing cost of microrobots used in biomedical field is relatively low and there is no safety concern about drug allergy. In addition, most of the microrobots used in the biomedical field are made of soft materials, which may avoid harms to the biological body. However, if the microrobot is used for thrombectomy to treat cerebral stroke, since the microrobot cannot easily output a mechanical force greater than a mechanical force of a device, the microrobot needs to spend more time to remove the thrombus, or an auxiliary surgery is required to shorten the time for treating cerebral stroke.

Therefore, it is an urgent problem to be solved in the art to develop a microrobot that can improve the mixing efficiency and dissolution efficiency in a specific area to shorten the treatment time for cerebral stroke.

### SUMMARY OF INVENTION

In order to solve the technical problems in the prior art described above, one object of the present disclosure is to provide a microrobot, which may achieve effects of enhancing mixing efficiency and dissolving efficiency of thrombus in a flow channel by placing the microrobot comprising polydimethylsiloxane and neodymium magnet particles on an electromagnet platform for manipulation.

Another object of the present disclosure is to provide a method for manufacturing a microrobot. A first acrylic mold and a second acrylic mold matched with each other are used to obtain the microrobot comprising polydimethylsiloxane and neodymium magnet particles. The object of enhancing mixing efficiency and dissolving efficiency of thrombus in a flow channel may be achieved by placing the microrobot on an electromagnet platform for manipulation.

In order to achieve the objects described above, the present disclosure provides a microrobot. The microrobot comprises:

a first block comprising polydimethylsiloxane;

a second block connected to one side of the first block, wherein the second block comprises a mixture, the mixture comprises polydimethylsiloxane and neodymium magnet particles, and wherein a weight ratio of the polydimethylsiloxane to the neodymium magnet particles of the second block is from 1:1 to 1:10 based on a total weight of the mixture of the second block; and

a third block connected to another side of the first block, wherein the third block and the first block are disposed oppositely, and the third block comprises the mixture, and wherein a weight ratio of the polydimethylsiloxane to the neodymium magnet particles of the third block is from 1:1 to 1:10 based on a total weight of the mixture of the third block.

In one embodiment, the neodymium magnet particles are neodymium iron boron (NdFeB) magnets.

In one embodiment, the second block and the third block have the same magnetization direction.

In one embodiment, the second block and the third block have different magnetization directions.

In one embodiment, the weight ratio of the polydimethylsiloxane to the neodymium magnet particles of the second block is 1:4.

In one embodiment, the weight ratio of the polydimethylsiloxane to the neodymium magnet particles of the third block is 1:4.

In one embodiment, a diameter of each of the neodymium magnet particles is between 0.5 μm and 50 μm.

In one embodiment, the microrobot has a length between 30 μm and 3000 μm, a width between 10 μm and 999 μm, and a height between 10 μm and 999 μm.

In one embodiment, the microrobot has a length of 100 μm, a width of 300 μm, and a height of 300 μm.

In one embodiment, the first block of the microrobot has a length between 10 μm and 999 μm, a width between 10 μm and 999 μm, and a height between 10 μm and 999 μm.

In one embodiment, the second block of the microrobot has a length between 10 μm and 999 μm, a width between 10 μm and 999 μm, and a height between 10 μm and 999 μm.

In one embodiment, the third block of the microrobot has a length between 10 μm and 999 μm, a width between 10 μm and 999 μm, and a height between 10 μm and 999 μm.

In one embodiment, a volume ratio of the first block, the second block to the third block is from 5 to 7:7 to 9:5 to 7.

In one embodiment, the microrobot further comprises a fourth block connected to the first block. The fourth block comprises the polydimethylsiloxane. The microrobot has a T-shaped structure.

In one embodiment, the microrobot further comprises:

a fifth block connected to the second block and the fourth block, wherein the fifth block comprises the mixture, and wherein a weight ratio of the polydimethylsiloxane to the neodymium magnet particles of the fifth block is from 1:1 to 1:10 based on a total weight of the mixture of the fifth block; and

a sixth block connected with the third block and the fourth block, wherein the fourth block is disposed between the fifth block and the sixth block, the sixth block comprises the mixture, and a weight ratio of the polydimethylsiloxane to the neodymium magnet particles of the sixth block is from 1:1 to 1:10 based on a total weight of the mixture of the sixth block.

In one embodiment, the second block, the third block, the fifth block, and the sixth block have the same magnetization direction with each other.

In one embodiment, the second block, the third block, the fifth block, and the sixth block have different magnetization directions with each other.

In one embodiment, the weight ratio of the polydimethylsiloxane to the neodymium magnet particles of the fifth block is 1:4.

In one embodiment, the weight ratio of the polydimethylsiloxane to the neodymium magnet particles of the sixth block is 1:4.

In one embodiment, the fourth block of the microrobot has a length between 10 μm and 999 μm, a width between 10 μm and 999 μm, and a height between 10 μm and 999 μm.

In one embodiment, the fifth block of the microrobot has a length between 10 μm and 999 μm, a width between 10 μm and 999 μm, and a height between 10 μm and 999 μm.

In one embodiment, the fifth block of the microrobot has a length between 10 μm and 999 μm, a width between 10 μm and 999 μm, and a height between 10 μm and 999 μm.

In one embodiment, the microrobot further comprises:

a seventh block connected to another side of the fourth block, wherein the seventh block and the first block are disposed oppositely, and the seventh block comprises the polydimethylsiloxane;

an eighth block connected to one side of the seventh block, wherein the eighth block comprises the mixture, and wherein a weight ratio of the polydimethylsiloxane to the neodymium magnet particles of the eighth block is from 1:1 to 1:10 based on a total weight of the mixture of the eighth block; and a ninth block connected to another side of the seventh block, wherein the seventh block is disposed between the eighth block and the ninth block, wherein the ninth block comprises the mixture; a weight ratio of the polydimethylsiloxane to the neodymium magnet particles of the ninth block is from 1:1 to 1:10 based on a total weight of the mixture of the ninth block; and wherein the microrobot has an H-shaped structure.

In one embodiment, the weight ratio of the polydimethylsiloxane to the neodymium magnet particles of the eighth block is 1:4.

In one embodiment, the weight ratio of the polydimethylsiloxane to the neodymium magnet particles of the ninth block is 1:4.

The present disclosure further provided a method of manufacturing a microrobot. The method comprises the steps of:

providing a first acrylic mold and a second acrylic mold, wherein the first acrylic mold has an inner wall, and the inner wall surrounds to form a first accommodating space; wherein the second acrylic mold has a U-shaped structure and the second acrylic mold matches the first accommodating space of the first acrylic mold; the second acrylic mold is provided with a first convex, a second convex, and a U-shaped recess, wherein the first convex is positioned at one end of the second acrylic mold and the second convex is positioned at another end of the second acrylic mold, and the U-shaped recess is formed between the first convex and the second convex;

injecting polydimethylsiloxane into the first accommodating space of the first acrylic mold;

placing the second acrylic mold in the first accommodating space of the first acrylic mold in a direction of facing the first convex and the second convex toward the first accommodating space of the first acrylic mold, allowing the first convex and the second convex of the second acrylic mold to extrude the polydimethylsiloxane out of the first accommodating space;

removing the second acrylic mold from the first accommodating space of the first acrylic mold after the polydimethylsiloxane being solidified to form a first block, wherein one side of the first block and the inner wall of the first acrylic mold surrounds to form a second accommodating space, and another side of the first block and the inner wall of the first acrylic mold surrounds to form a third accommodating space;

mixing the polydimethylsiloxane and neodymium magnet particles in a weight ratio of 1:1 to 1:10 to form a mixture, and injecting the mixture into the second accommodating space of the first acrylic mold;

after the mixture being solidified in the second accommodating space to form a second block, magnetizing the second block, wherein the second block connects to one side of the first block;

injecting the mixture into the third accommodating space of the first acrylic mold;

after the mixture being solidified in the third accommodating space to form a third block, magnetizing the third block, wherein the third block connects to another side of the first block, and the third block and the second block are disposed oppositely; and taking out the first block, the second block, and the third block from the first acrylic mold to obtain the microrobot.

In one embodiment, the weight ratio of the polydimethylsiloxane to the neodymium magnet particles of the second block is 1:4.

In one embodiment, the weight ratio of the polydimethylsiloxane to the neodymium magnet particles of the third block is 1:4.

In one embodiment, the microrobot has a length between 30 μm and 3000 μm, a width between 10 μm and 999 μm, and a height between 10 μm and 999 μm.

In one embodiment, after the step of injecting the polydimethylsiloxane into the first accommodating space of the first acrylic mold, the method further comprises a step of removing the polydimethylsiloxane beyond the first accommodating space of the first acrylic mold.

In one embodiment, after the step of injecting the mixture into the second accommodating space of the first acrylic mold, the method further comprises a step of removing the mixture beyond the second accommodating space of the first acrylic mold.

In one embodiment, after the step of injecting the mixture into the third accommodating space of the first acrylic mold, the method further comprises a step of removing the mixture beyond the third accommodating space of the first acrylic mold.

A microrobot manufactured by a method of manufacturing the microrobot of the present disclosure comprises several blocks containing neodymium magnet particles. Each block containing the neodymium magnet particles may be magnetized separately, so that each block containing the neodymium magnet particles has the same or different magnetization directions. When using the microrobot, the microrobot may be placed on an electromagnet platform, and the microrobot may move and rotate precisely in an environment of microscale flow channel through magnetic drive, followed by generating fluid vortex to enhance the mixing efficiency and dissolution efficiency in a specific area. Therefore, the microrobot of the present disclosure may be applied to the treatment of cerebral stroke by dissolving the thrombus. The thrombus structure may become loose through the fluid vortex such as a blood vortex generated by the magnetic drive, and the mixing of anticoagulant drugs and thrombi in the circulation may be enhanced for thrombolytic therapy.

### BRIEF DESCRIPTION OF DRAWINGS

In order to explain the technical solutions of the present disclosure more clearly, the following will briefly introduce the drawings used in the description of the embodiments or the related art. Obviously, the drawings described below are only some embodiments of the present disclosure. For those skilled in the art, other drawings can be obtained based on these drawings without making creative efforts.

FIG. 1 is a step flow chart of a method of manufacturing a microrobot of the present disclosure.

FIG. 2 is a schematic flow chart of a method of manufacturing a microrobot of the present disclosure.

FIG. 3A is a schematic view of a stereoscopic structure of a first microrobot of the present disclosure.

FIG. 3B is a top view of a microphotograph of the first microrobot of the present disclosure.

FIG. 4 is a schematic view of a stereoscopic structure of a second microrobot of the present disclosure.

FIG. 5 is a schematic view of a stereoscopic structure of a third microrobot of the present disclosure

FIG. 6 is a schematic view of a stereoscopic structure of a fourth microrobot of the present disclosure.

FIG. 7 is a top view of a photo of an electromagnet platform that drives the movement of the microrobot of the present disclosure.

FIG. 8A is a schematic structural view of an open flow channel for examining the efficiency of the microrobot of the present disclosure.

FIG. 8B is a schematic structural view of a closed flow channel for examining the efficiency of the microrobot of the present disclosure.

FIG. 9A is a graph of three different waveforms of output signals of the graphic programming language LabVIEW that drives the movement of the microrobot of the present disclosure.

FIG. 9B is displacement curves and a microphotograph of the microrobot of the present disclosure in a x-direction under the control of three different waveform signals and the rotation frequency of magnetic field with 9 Hz.

FIG. 9C is displacement curves and a microphotograph of the microrobot of the present disclosure in a y-direction under the control of three different waveform signals and the rotation frequency of magnetic field with 9 Hz.

FIG. 10A is displacement curves and a microphotograph of the microrobot of the present disclosure in a x-direction under the control of sinewave signal and the rotation frequency of magnetic field with 3 Hz, 6 Hz, 9 Hz, 12 Hz, and 15 Hz.

FIG. 10B is displacement curves and a microphotograph of the microrobot of the present disclosure in a y-direction under the control of sinewave signal and the rotation frequency of magnetic field with 3 Hz, 6 Hz, 9 Hz, 12 Hz, and 15 Hz.

FIG. 11 is a graph of the mixing efficiency of the microrobot of the present disclosure under the motion modes of Mode I, Mode II, and Mode III and a schematic diagram of the displacement trajectory of the microrobot.

FIG. 12A is a graph of the shrinkage rate of sodium chloride crystals dissolving sodium chloride crystals by the microrobot of the present disclosure in the open flow channel under the motion modes of mode I, mode II, and mode III and a microphotograph under 0 second and 200 second.

FIG. 12B is a graph of the shrinkage rate of sodium chloride crystals dissolving sodium chloride crystals by the microrobot of the present disclosure in the closed flow channel under the motion modes of mode I, mode II, and mode III and a microphotograph under 0 second and 200 second.

### DETAILED DESCRIPTION OF PREFERRED EMBODIMENTS

The following describes the embodiment of the present disclosure through specific examples. Those skilled in the field can understand other advantages and effects of the present disclosure from the content disclosed in the present specification. However, the exemplary embodiments disclosed in the present disclosure are merely for illustrative purposes and should not be construed as a limiting the scope of the present disclosure. In other words, the present disclosure can also be implemented or applied by other different specific embodiments, and various details in the present specification can also be modified and changed based on different viewpoints and applications without departing from the concept of the present disclosure.

Unless otherwise described herein, the singular forms “a” and “the” used in the specification and the appended claims of the present disclosure comprise plural entities. Unless otherwise described herein, the term “or” used in the specification and the appended claims of the present disclosure comprises the meaning of “and/or”.

### Preparation Example 1: Preparation of a First Microrobot

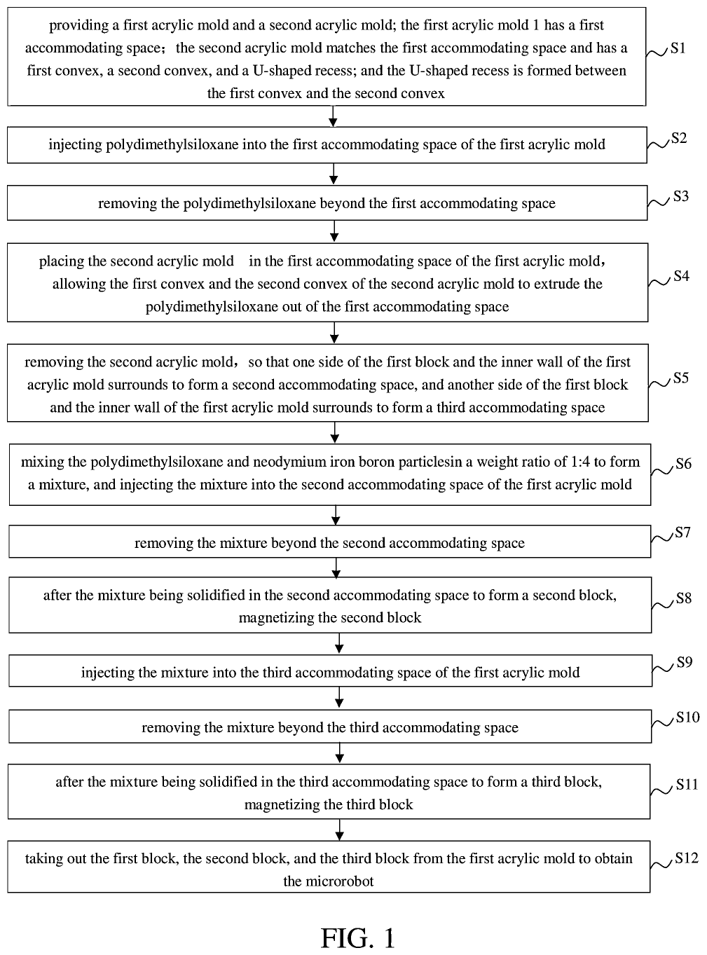

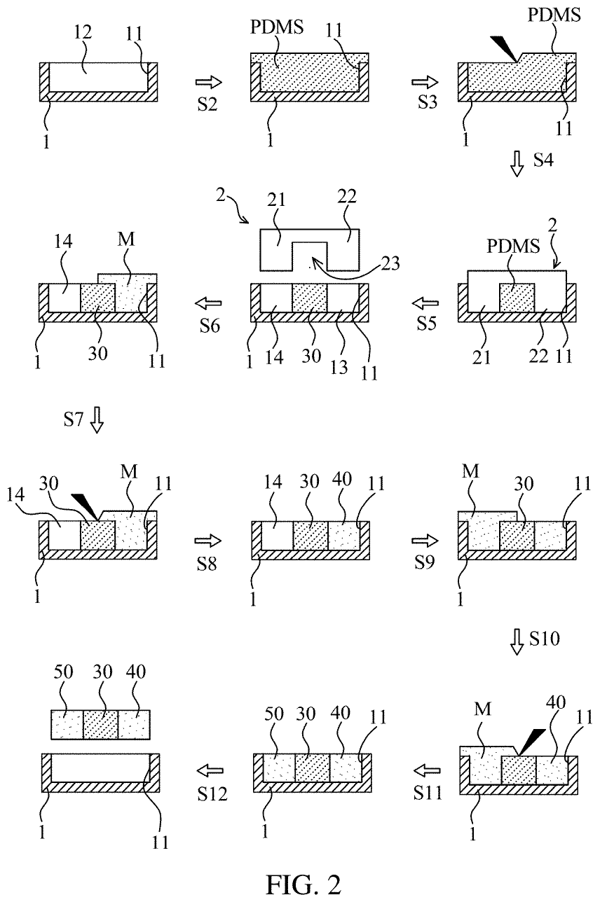

Referring to FIG. 1 and FIG. 2, a method of manufacturing a microrobot comprises the steps of:

Step S1: providing a first acrylic mold 1 and a second acrylic mold 2. The first acrylic mold 1 has an inner wall 11, and the inner wall 11 surrounds to form a first accommodating space 12. The second acrylic mold 2 has a U-shaped structure and the second acrylic mold 2 matches the first accommodating space 12 of the first acrylic mold 1. The second acrylic mold 2 is provided with a first convex 21, a second convex 22, and a U-shaped recess 23. The first convex 21 is positioned at one end of the second acrylic mold 2 and the second convex 22 is positioned at another end of the second acrylic mold 2. The U-shaped recess 23 is formed between the first convex 21 and the second convex 22;

Step S2: injecting polydimethylsiloxane (PDMS, Sylgard 184) into the first accommodating space 12 of the first acrylic mold 1;

Step S3: removing the polydimethylsiloxane beyond the first accommodating space 12 by using a scraper;

Step S4: placing the second acrylic mold 2 in the first accommodating space 12 of the first acrylic mold 1 in a direction of facing the first convex 21 and the second convex 22 toward the first accommodating space 12 of the first acrylic mold 1, allowing the first convex 21 and the second convex 22 of the second acrylic mold 2 to extrude the polydimethylsiloxane out of the first accommodating space 1;

Step S5: removing the second acrylic mold 2 from the first accommodating space 12 of the first acrylic mold 1 after the polydimethylsiloxane being solidified to form a first block 30, wherein one side of the first block 30 and the inner wall 11 of the first acrylic mold 1 surrounds to form a second accommodating space 13, and another side of the first block 30 and the inner wall 11 of the first acrylic mold 1 surrounds to form a third accommodating space 14;

Step S6: mixing the polydimethylsiloxane and neodymium iron boron (NdFeB) particles (MQP-15-7) in a weight ratio of 1:4 to form a mixture M, and injecting the mixture M into the second accommodating space 13 of the first acrylic mold 1;

Step S7: removing the mixture M beyond the second accommodating space 13 by using the scraper;

Step S8: after the mixture M being solidified in the second accommodating space 13 to form a second block 40, magnetizing the second block 40, wherein the second block 40 connects to one side of the first block 30;

Step S9: injecting the mixture M into the third accommodating space 14 of the first acrylic mold 1;

Step S10: removing the polydimethylsiloxane beyond the third accommodating space 14 by using the scraper;

Step S11: after the mixture M being solidified in the third accommodating space 14 to form a third block 50, magnetizing the third block 50, wherein the third block 50 connects to another side of the first block 30; the third block 50 and the second block 40 are disposed oppositely, and wherein the third block 50 and the second block 40 have different magnetization directions with each other; and

Step S12: taking out the first block 30, the second block 40, and the third block 50 from the first acrylic mold 1 to obtain the first microrobot.

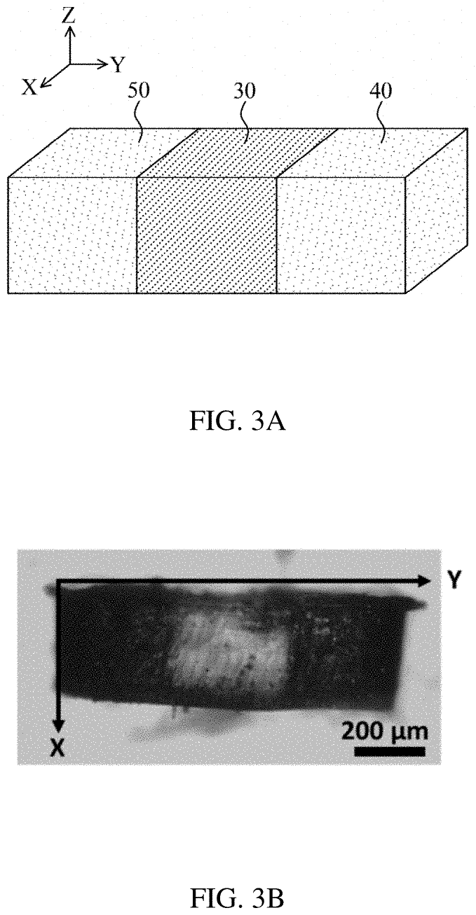

Referring to FIG. 3A, the first microrobot manufactured by Preparation Example 1 comprises the first block 30, the second block 40, and the third block 50. The first block 30, the second block 40, and the third block 50 are connected to each other, and the first block 30 is disposed between the second block 40 and the third block 50. Referring to FIG. 3B, the first microrobot is observed under a microscope. An overall structure of the first microrobot is a cuboid with a length of 30 μm to 3000 μm, a width of 10 μm to 999 μm, and a height of 10 μm to 999 μm. Preferably, the length is 1000 μm, the width is 300 μm, and the height is 300 μm, so that the first microrobot may translate and rotate in a cerebral artery with a diameter of 2 mm.

### Preparation Example 2: Preparation of a Second Microrobot

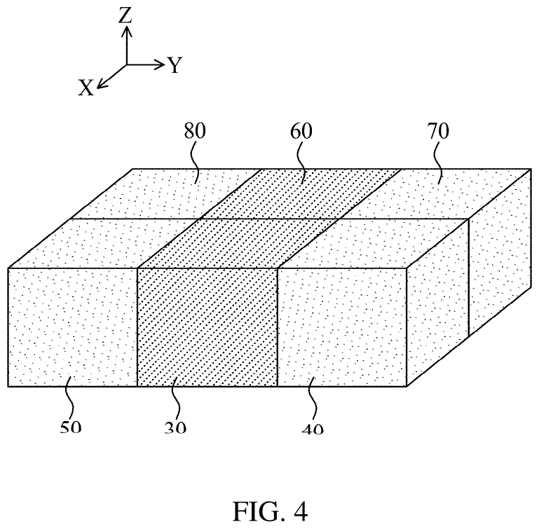

A method of manufacturing the second microrobot is similar to the method of Preparation Example 1. The difference between Preparation Example 2 and Preparation Example 1 is that volumes of a first acrylic mold and a second acrylic mold used in Preparation Example 2 are twice the volumes of the first acrylic mold and the second acrylic mold used in Preparation Example 1. The second microrobot as shown in FIG. 4 prepared by Preparation Example 2 comprises a first block 30, a second block 40, a third block 50, a fourth block 60, a fifth block 70, and a sixth block 80. The first block 30, the second block 40, and the third block 50 are connected to each other, and the block 30 is disposed between the second block 40 and the third block 50. The fourth block 60 is connected to the first block 30. The fifth block 70 is connected to the second block 40 and the fourth block 60. The sixth block 80 is connected to the third block 50 and the fourth block 60, and the fourth block 60 is disposed between the fifth block 70 and the sixth block 80.

The fourth block 60 comprises polydimethylsiloxane. The fifth block 70 comprises a mixture M, and a weight ratio of the polydimethylsiloxane to neodymium iron boron of the fifth block 70 is 1:4 based on a total weight of the mixture M of the fifth block 70. The sixth block 80 comprises the mixture M, and a weight ratio of the polydimethylsiloxane to neodymium iron boron of the sixth block 80 is 1:4 based on a total weight of the mixture M of the sixth block 80. In addition, the second block 40, the third block 50, the fifth block 70, and the sixth block 80 have the same magnetization direction or different magnetization directions with each other.

### Preparation Example 3: Preparation of a Third Microrobot

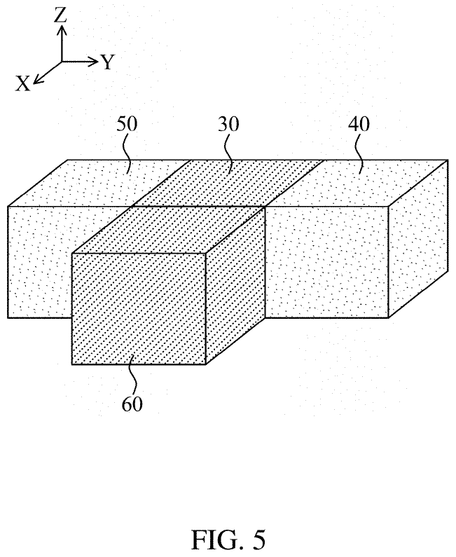

A method of manufacturing the third microrobot is similar to the method of Preparation Example 1. The difference between Preparation Example 3 and Preparation Example 1 is that the third microrobot as shown in FIG. 5 prepared by Preparation Example 3 has a T-shaped structure. The third microrobot comprises a first block 30, a second block 40, a third block 50, and a fourth block 60. The first block 30, the second block 40, and the third block 50 are connected to each other, and the first block 30 is disposed between the second block 40 and the third block 50. The fourth block 60 is connected to the first block 30. In addition, the second block 40 and the third block 50 have the same magnetization direction or different magnetization directions.

### Preparation Example 4: Preparation of a Fourth Microrobot

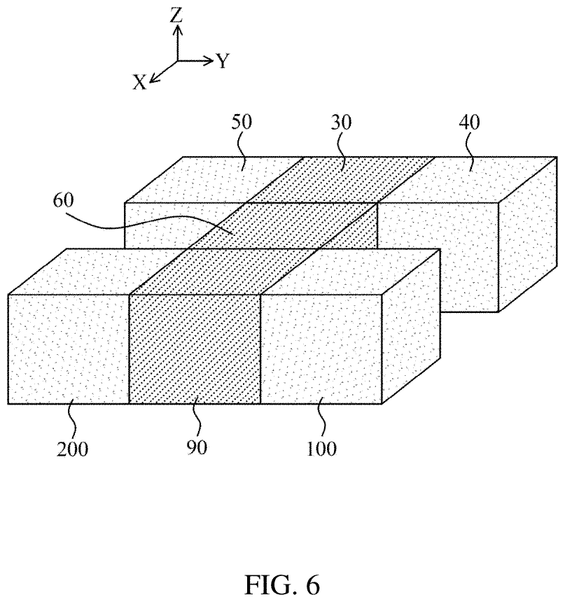

A method of manufacturing the fourth microrobot is similar to the method of Preparation Example 1. The difference between Preparation Example 4 and Preparation Example 1 is that the fourth microrobot as shown in FIG. 6 prepared by Preparation Example 4 has a H-shaped structure. The fourth microrobot comprises a first block 30, a second block 40, a third block 50, a fourth block 60, a seventh block 90, an eighth block 100, and a ninth block 200. The first block 30, the second block 40, and the third block 50 are connected to each other, and the first block 30 is disposed between the second block 40 and the third block 50. The fourth block 60 is connected to the first block 30. The seventh block 90 is connected to the fourth block 60, and the seventh block 90 and the first block 30 are disposed oppositely. The eighth block 100 is connected to one side of the seventh block 90. The ninth block 200 is connected to another side of the seventh block 90, and the seventh block 90 is disposed between the eighth block 100 and the ninth block 200.

The seventh block 90 comprises polydimethylsiloxane. The eighth block 100 comprises a mixture M, and a weight ratio of the polydimethylsiloxane to neodymium iron boron of the eighth block 100 is 1:4 based on a total weight of the mixture M of the eighth block 100. The ninth block 200 comprises the mixture M, and a weight ratio of the polydimethylsiloxane to neodymium iron boron of the ninth block 200 is 1:4 based on a total weight of the mixture M of the ninth block 200. In addition, the second block 40, the third block 50, the eighth block 100, and the ninth block 200 have the same magnetization direction or different magnetization directions with each other.

### Preparation Example 5: Preparation of an Electromagnet Platform

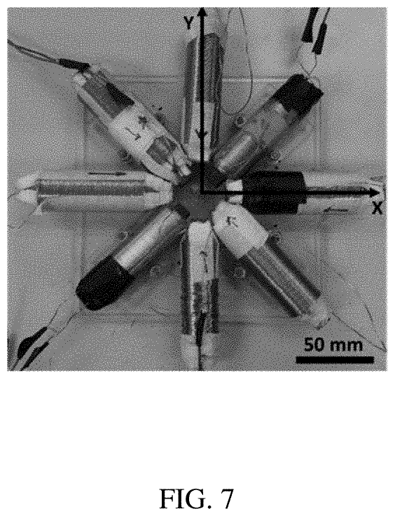

Referring to FIG. 7, the electromagnet platform comprises eight electromagnet coils. Each electromagnet coil is made by winding an enameled wire on a rectangular copper strip, and the total number of turns of each electromagnet coil is 1200 turns. A magnetic field strength of each electromagnet coil is calculated by formula (1), where B is a magnetic flux density; μᵣ is a relative permeability; to is a vacuum permeability; I is a current passing through the wire, and N is a number of turns of the wire per unit length. Using the relative permeability of carbon steel with a value of 100 and a measured peak current of a single coil with a value of 0.3 A, a calculated peak strength of a single electromagnet coil is about 500 mT.

B=μᵣμ₀IN Formula (1)

### Preparation Example 6: Preparation of an Open Flow Channel and a Closed Flow Channel

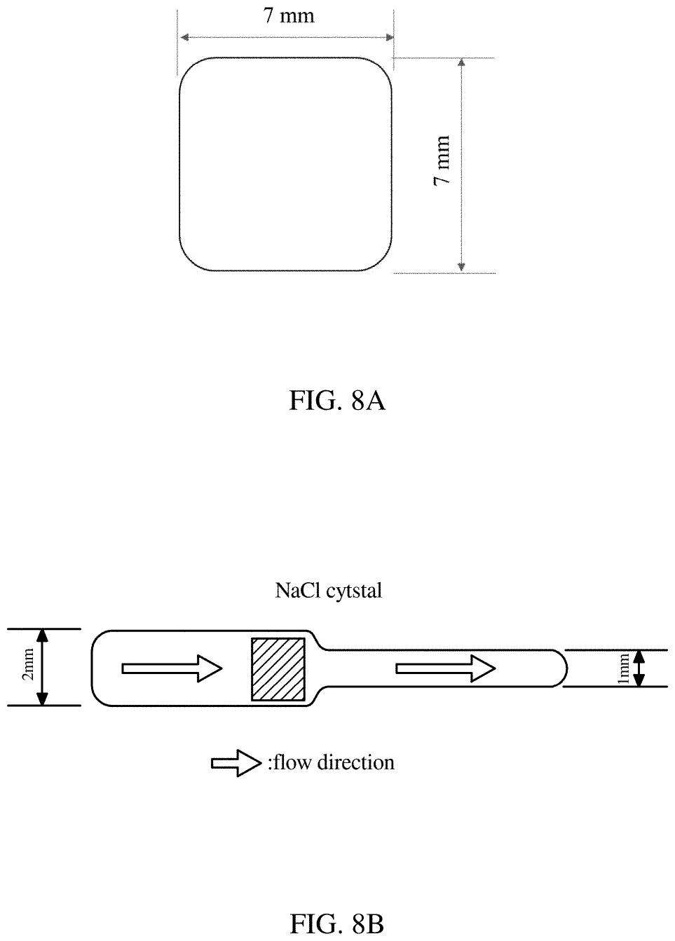

Referring to FIG. 8A, a water tank with a size of 7 mm (length)×7 mm (width)×1 mm (depth) is used as the open flow channel. Referring to FIG. 8B, the closed flow channel is designed to taper from 2 mm to 1 mm in width and the depth of the closed flow channel is 2 mm based on a geometric dimensions of the human cerebral artery.

Moreover, a 75 wt % glycerol aqueous solution with a density and viscosity similar to a density and viscosity of human blood is used to flow in the open flow channel and the closed flow channel as a blood-like fluid to optimize a dynamic control of the first microrobot and examine the mixing efficiency of the microrobot. Furthermore, deionized water is used to flow in the open flow channel and the closed flow channel to perform a test of the dissolution efficiency of the microrobot.

### Example 1: Optimizing the Dynamic Control of the Microrobot

A Data Acquisition (NI cDAQ-9174) (purchased from National Instruments Corporation, Austin, Tex., USA) and embedded signal input and output modules (NI 9201 and NI 9264) is connected to the electromagnet coils and an external power supply for dynamic control of the microrobot. Using a graphical programming language LabVIEW (purchased from National Instruments Corporation, Austin, Tex., USA) to establish a computer operation interface to modify control parameters such as the rotation frequency and strength of the magnetic field. The efficiency of the first microrobot under different control parameters may be detected.

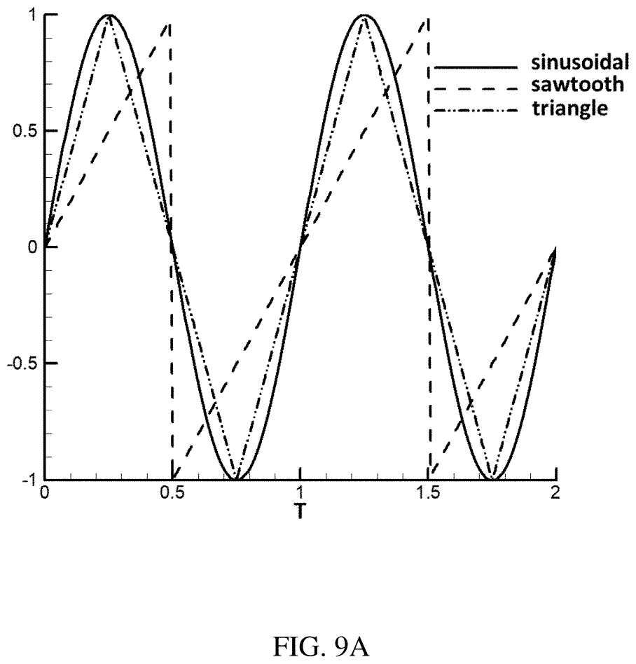

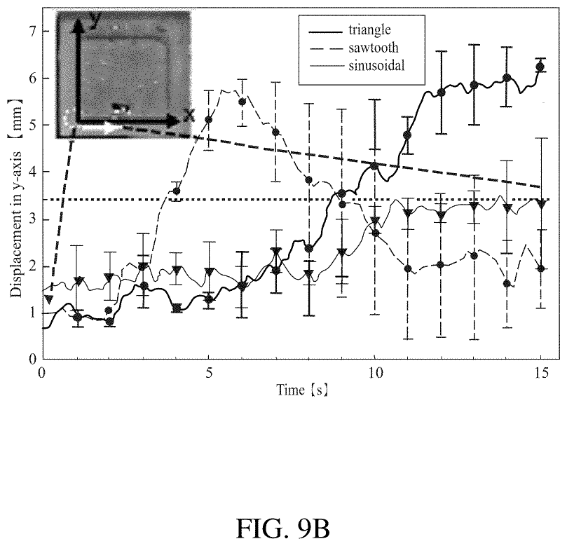

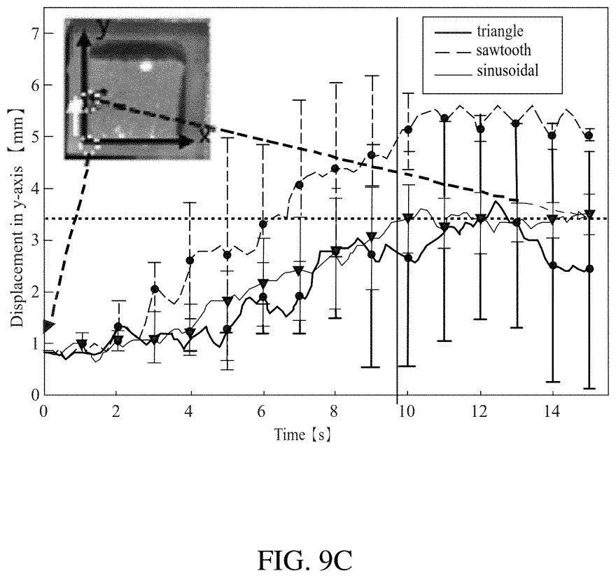

The present embodiment detects the efficiency of the first microrobot under three different types of waveform signals. As shown in FIG. 9A, the three different types of waveform signals comprise a sawtooth wave, a triangle wave, and a sinusoidal wave. The sawtooth wave has a signal steep drop in each cycle, and is a smooth curve. The triangle wave is composed of two oblique straight lines. The waveforms of the sawtooth wave, the triangle wave, and the sinusoidal wave respectively represent the trend of electromagnet change: steep drop in intensity, rise and fall in constant rate, and smooth curve. Thus, these waveforms may be used to change the strength of each electromagnet, for example, a transition from a steep drop to a smooth curve. Under the control of three different waveform signals and different magnetic field rotation frequencies of the output signal of the graphical programming language Lab VIEW (purchased from National Instruments Corporation, Austin, Tex., USA), the dynamics of the first microrobot is tracked, and the test is repeated.

FIG. 9B and FIG. 9C respectively show the displacement curves and microphotographs of the first microrobot in a x-direction and a y-direction under the control of three different waveform signals and a magnetic field rotation frequency of 9 Hz. The results show that under the control of the sawtooth wave signal and the triangular wave signal, the displacements of the first microrobot in the x-direction and the y-direction are overshoot or fall back, and the path characteristics in the x-direction and the y-direction are also inconsistent. Under the control of the sinewave signal and the magnetic field rotation frequency of 9 Hz, the displacements of the first microrobot in the x-direction and the y-direction reach a stable dynamic after 10 seconds. Accordingly, the sinewave signal is used to perform the following experiments on the trajectory path of first microrobot.

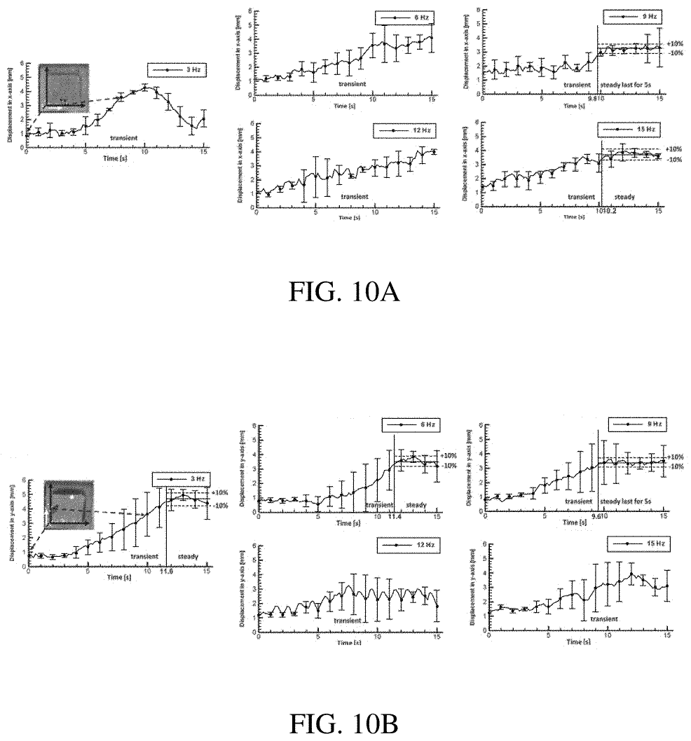

The displacement trajectory and dynamic stability of the first microrobot under the control of the sinewave signal and different magnetic field rotation frequencies (3 Hz, 6 Hz, 9 Hz, 12 Hz, and 15 Hz) are detected, while considering the displacement error (not exceeding the average value±10%) to evaluate the performance of the microrobot. As shown in FIG. 10A and FIG. 10B, the results show that under the control of the sinewave signal and the magnetic field rotation frequencies of 3 Hz, 6 Hz, and 12 Hz, the displacements of the first microrobot in the x-direction and the y-direction are obviously in transient state within 15 seconds, and there is no tendency to maintain in a specific position. Moreover, under the control of the sinewave signal and the magnetic field rotation frequency of 15 Hz, the displacement of the first microrobot in the x-direction may maintain a small vibration after 10.2 seconds. However, displacements in the y-direction are obviously in transient state. By contrast, under the control of the sinewave signal and the magnetic field rotation frequency of 9 Hz, the first microrobot may move to a target position, and the displacements in the x-direction and y-direction may remain stable for more than 5 seconds. Moreover, the oscillation of the average displacement does not exceed 0.3 mm of the average. The results show that compared with other magnetic field rotation frequencies, the first microrobot under the control of the sinewave signal and the magnetic field rotation frequency of 9 Hz has good stable efficiency.

### Example 2: Examining the Mixing Efficiency of the First Microrobot

Under three different motion modes (mode I, mode II, and mode III), the mixing efficiency of the first microrobot is examined. Mode I (without the first microrobot) is used as a control group. Mode II (static rotation) allows the first microrobot to be maintained at an original position (i.e., at the lower left corner of the flow channel). Mode III (rotation with translation) allows the first microrobot to move with rotation in the flow channel. Moreover, the mixing efficiency (%) of the first microrobot in the flow channel is calculated by formula (2),

$$Mixing efficiency ( \% ) = ( 1 - \frac{1}{\overline{m}} \sqrt{\frac{{Σ}_{i}^{n} {( {m}_{i} - \overline{m} )}^{2}}{n}} ) \times 100 , \qquad Formula ( 2 )$$

where mᵢ represents an intensity of a pixel, which is the brightness; m̅ represents an average intensity of all pixels in an image, which is used to measure an uniformity of the fluid; and n represents a total number of pixels in the image.

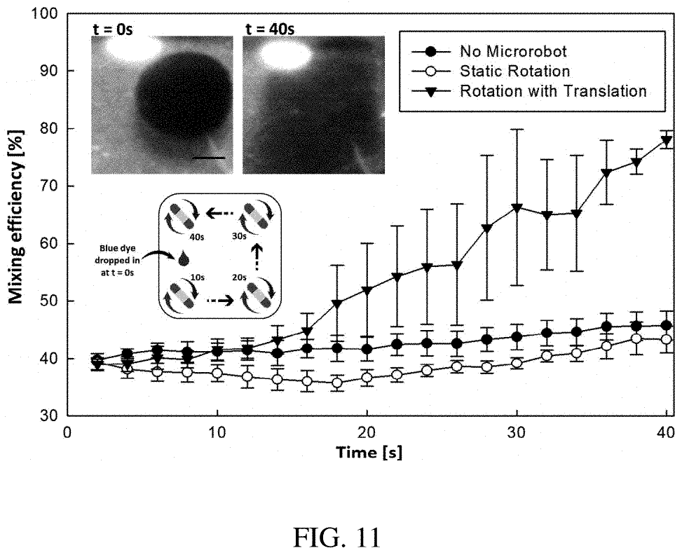

The results are shown in FIG. 11. Initially at time 0 second, a blue dye is clearly observed with distinct boundaries in the blood-like fluid (static state). In addition, there is no significant change in the mixing efficiency in the first 10 seconds in Mode I, Mode II, and Mode III. However, from 10 seconds to 40 seconds, the mixing efficiency of the first microrobot in the motion mode of Mode III significantly increases from 40% to 80%, while the maximum mixing efficiency of Mode I and Mode II is merely between 39% and 42%.

Furthermore, by calculating the slope of the mixing efficiency curve from 10 seconds to 40 seconds, the mixing efficiency of the first microrobot in the motion modes of Mode I, Mode II, and Mode III may be quantified. The time required for mode III (rotation with translation) to reach the highest mixing efficiency was 5.18 times faster than the time required for mode II (static rotation) to reach the highest mixing efficiency. The above results clearly show that the first microrobot may enhance the mixing efficiency in a specific area by using the motion mode of rotation with translation on the boundary of the flow channel.

### Example 3: Detecting the Dissolution Efficiency of Micro-Robots

In order to examine the dissolution efficiency of the first microrobot, the first microrobot is placed in the open flow channel and the closed flow channel under three different motion modes (mode I, mode II, and mode III) for the dissolution test of sodium chloride crystals. The shrinkage percentage (%) of the sodium chloride crystal, which represents a ratio of an instant area of the sodium chloride crystal to an initial area of the sodium chloride crystal is calculated by Formula (3).

$$Shrinking percentage = ( 1 - \frac{{n}_{t}}{{n}_{0}} ) \times 100 , \qquad Formula ( 3 )$$

wherein nₜ and n₀ represent the number of pixels that the sodium chloride crystal took at time=t second and time=0 second, respectively. In addition, a rate of shrinkage percentage is calculated by formula (4) as a parameter for evaluating the function of the first microrobot,

$$Rate of shrinkage percentage = \frac{shrinkage percentage}{Δ t} ( \% / s ) , \qquad Formula ( 4 )$$

where t represents time.

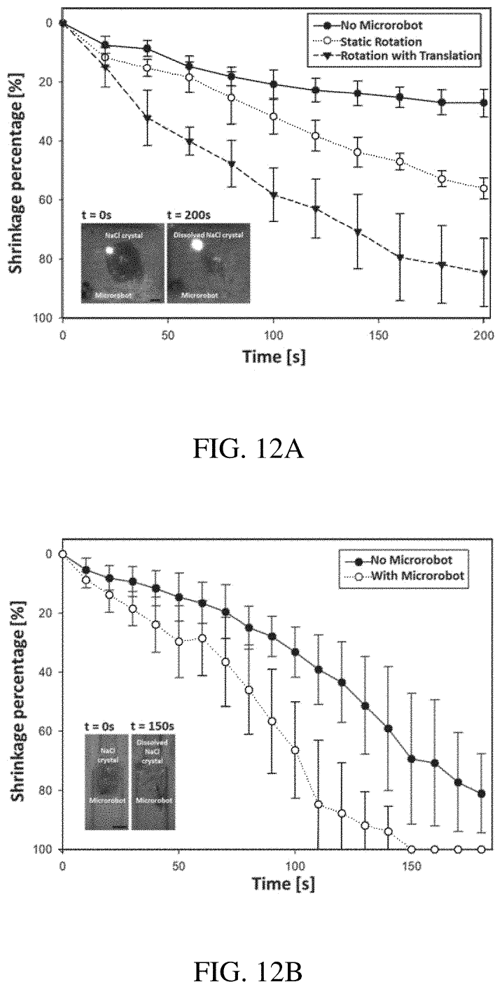

FIG. 12A shows a graph of the shrinkage rate of the sodium chloride crystal after dissolving the sodium chloride crystal by the first microrobot in the motion modes of mode I, mode II, and mode III in the open flow channel, and the microphotograph taken at t=0 second and t=200 second. The results show that in the motion mode of mode III, the shrinkage rate of the first microrobot dissolving the sodium chloride crystal may reach 84.5% at t=200 second. During the dissolution process, the four sides of the rectangle of the sodium chloride crystal initially shrink, followed by forming a star-like shape and finally dissolves into an irregular shape. In contrast, in the motion modes of Mode I and Mode II, the shrinkage rate of the first microrobot dissolving the sodium chloride crystal is only about 27.1% and 56%, respectively at t=200 second.

Moreover, in the motion modes of Mode I and Mode II, average values of the rate of shrinkage percentage of the first microrobot dissolving the sodium chloride crystal (at t=0 second to 200 second) are 0.136% and 0.280% per second, respectively. In the motion mode of Mode III, an average value of the rate of shrinkage percentage of the first microrobot dissolving the sodium chloride crystal (at t=0 second to 200 second) is 0.422% per second.

FIG. 12B shows a graph of the shrinkage rate of the sodium chloride crystal after dissolving the sodium chloride crystal by the first microrobot in the closed flow channel in the motion mode of Mode II, and the microphotograph taken at t=0 second and t=180 second. The results show that in the absence of the first microrobot, the shrinkage rate of the sodium chloride crystal is about 81.1% (at t=180 second), while in the motion mode of Mode II, the first microrobot dissolves the sodium chloride crystal when the time is 150 seconds. The shrinkage rate of the sodium chloride crystal after dissolving the sodium chloride crystal by the first microrobot is 100% (at t=150 second).

Moreover, in the absence of the first microrobot, the average value of the rate of shrinkage percentage of the first microrobot dissolving the sodium chloride crystal (at t=180 second) is 0.450% per second. In the motion mode of the Mode II, the average value of the rate of shrinkage percentage of the first microrobot dissolving the sodium chloride crystal (at t=150 second) 0.667% per second.

The results mentioned above show that the first microrobot may move accurately in an environment of microscale flow channels and may achieve the efficiencies of smoothing the fluid in the open flow channel and closed flow channel, and accelerating the dissolution of substances in the fluid. In addition, in the open channel, the dissolution rate of the substance may be increased by three fold, and in the closed channel, the dissolution rate of the substance may be increased by about 50%.

Based on the above results, the first microrobot prepared in Preparation Example 1 may be placed on the electromagnet platform, and may move precisely in the environment of the microscale flow channel through magnetic drive, followed by generating fluid vortex to enhance the mixing efficiency and dissolution efficiency in a specific area.

Moreover, both of the second microrobot prepared in Preparation Example 2 and the fourth microrobot prepared in Preparation Example 4 comprise four blocks containing neodymium magnet particles, and each block containing neodymium magnet particles may be respectively magnetized so that each block containing the neodymium magnet particles has the same magnetization direction or different magnetization directions. It allows to generate different dynamic behaviors on the electromagnet platform.

Furthermore, the second microrobot prepared in Preparation Example 2, the third microrobot prepared in Preparation Example 3, and the fourth microrobot prepared in Preparation Example 4 may be placed on the electromagnet platform and may move precisely in the environment of the microscale flow channel through magnetic drive, followed by generating fluid vortex to enhance the mixing efficiency and dissolution efficiency in a specific area (data not shown).

From the above, the microrobot prepared by the present disclosure may be applied to the treatment of thrombus dissolving in cerebral stroke. The thrombus structure may become loose through the fluid vortex such as a blood vortex generated by the magnetic drive, and the mixing of anticoagulant drugs and thrombi in the circulation area may be enhanced for thrombolytic therapy.

The above provides a detailed introduction to the implementation of the present disclosure, and specific examples are used herein to describe the principles and implementations of the present disclosure, and the description of the implementations above is merely used to help understand the present disclosure. Moreover, for those skilled in the art, according to a concept of the present disclosure, there will be changes in the specific embodiment and the scope of present disclosure. In summary, the content of the specification should not be construed as a limitation to the present disclosure.

What is claimed is:

1. A microrobot, comprising:
a first block comprising polydimethylsiloxane;
a second block connected to one side of the first block, wherein the second block comprises a mixture, the mixture comprises polydimethylsiloxane and neodymium magnet particles, and wherein a weight ratio of the polydimethylsiloxane to the neodymium magnet particles of the second block is from 1:1 to 1:10 based on a total weight of the mixture of the second block; and
a third block connected to another side of the first block, wherein the third block and the first block are disposed oppositely, and the third block comprises the mixture, and wherein a weight ratio of the polydimethylsiloxane to the neodymium magnet particles of the third block is from 1:1 to 1:10 based on a total weight of the mixture of the third block.

2. The microrobot according to claim 1, wherein the second block and the third block have the same magnetization direction.

3. The microrobot according to claim 1, wherein the second block and the third block have different magnetization directions.

4. The microrobot according to claim 1, wherein the weight ratio of the polydimethylsiloxane to the neodymium magnet particles of the second block is 1:4.

5. The microrobot according to claim 4, wherein the weight ratio of the polydimethylsiloxane to the neodymium magnet particles of the third block is 1:4.

6. The microrobot according to claim 1, wherein the microrobot has a length between 30 μm and 3000 μm, a width between 10 μm and 999 μm, and a height between 10 μm and 999 μm.

7. The microrobot according to claim 1, wherein the first block of the microrobot has a length between 10 μm and 999 μm, a width between 10 μm and 999 μm, and a height between 10 μm and 999 μm.

8. The microrobot according to claim 1, wherein the second block of the microrobot has a length between 10 μm and 999 μm, a width between 10 μm and 999 μm, and a height between 10 μm and 999 μm.

9. The microrobot according to claim 1, wherein the third block of the microrobot has a length between 10 μm and 999 μm, a width between 10 μm and 999 μm, and a height between 10 μm and 999 μm.

10. The microrobot according to claim 1, wherein the microrobot further comprises a fourth block connected to the first block, the fourth block comprises the polydimethylsiloxane, and the microrobot has a T-shaped structure.

11. The microrobot according to claim 10, wherein the microrobot further comprises:
a fifth block connected to the second block and the fourth block, wherein the fifth block comprises the mixture, and wherein a weight ratio of the polydimethylsiloxane to the neodymium magnet particles of the fifth block is from 1:1 to 1:10 based on a total weight of the mixture of the fifth block; and
a sixth block connected with the third block and the fourth block, wherein the fourth block is disposed between the fifth block and the sixth block, the sixth block comprises the mixture, and a weight ratio of the polydimethylsiloxane to the neodymium magnet particles of the sixth block is from 1:1 to 1:10 based on a total weight of the mixture of the sixth block.

12. The microrobot according to claim 11, wherein the second block, the third block, the fifth block, and the sixth block have the same magnetization direction with each other.

13. The microrobot according to claim 11, wherein the second block, the third block, the fifth block, and the sixth block have different magnetization directions with each other.

14. The microrobot according to claim 11, wherein the weight ratio of the polydimethylsiloxane to the neodymium magnet particles of the fifth block is 1:4.

15. The microrobot according to claim 14, wherein the weight ratio of the polydimethylsiloxane to the neodymium magnet particles of the sixth block is 1:4.

16. The microrobot according to claim 10, wherein the microrobot further comprises:
a seventh block connected to another side of the fourth block, wherein the seventh block and the first block are disposed oppositely, and the seventh block comprises the polydimethylsiloxane;
an eighth block connected to one side of the seventh block, wherein the eighth block comprises the mixture, and wherein a weight ratio of the polydimethylsiloxane to the neodymium magnet particles of the eighth block is from 1:1 to 1:10 based on a total weight of the mixture of the eighth block; and
a ninth block connected to another side of the seventh block, wherein the seventh block is disposed between the eighth block and the ninth block, wherein the ninth block comprises the mixture; a weight ratio of the polydimethylsiloxane to the neodymium magnet particles of the ninth block is from 1:1 to 1:10 based on a total weight of the mixture of the ninth block; and wherein the microrobot has an H-shaped structure.

17. The microrobot according to claim 10, wherein a diameter of each of the neodymium magnet particles is between 0.5 μm and 50 μm.

18. A method of manufacturing a microrobot, comprising the steps of:
providing a first acrylic mold and a second acrylic mold, wherein the first acrylic mold has an inner wall, and the inner wall surrounds to form a first accommodating space; wherein the second acrylic mold has a U-shaped structure and the second acrylic mold matches the first accommodating space of the first acrylic mold; the second acrylic mold is provided with a first convex, a second convex, and a U-shaped recess, wherein the first convex is positioned at one end of the second acrylic mold and the second convex is positioned at another end of the second acrylic mold, and the U-shaped recess is formed between the first convex and the second convex;
injecting polydimethylsiloxane into the first accommodating space of the first acrylic mold;
placing the second acrylic mold in the first accommodating space of the first acrylic mold in a direction of facing the first convex and the second convex toward the first accommodating space of the first acrylic mold, allowing the first convex and the second convex of the second acrylic mold to extrude the polydimethylsiloxane out of the first accommodating space;
removing the second acrylic mold from the first accommodating space of the first acrylic mold after the polydimethylsiloxane being solidified to form a first block, wherein one side of the first block and the inner wall of the first acrylic mold surrounds to form a second accommodating space, and another side of the first block and the inner wall of the first acrylic mold surrounds to form a third accommodating space;
mixing the polydimethylsiloxane and neodymium magnet particles in a weight ratio of 1:1 to 1:10 to form a mixture, and injecting the mixture into the second accommodating space of the first acrylic mold;
after the mixture being solidified in the second accommodating space to form a second block, magnetizing the second block, wherein the second block connects to one side of the first block;
injecting the mixture into the third accommodating space of the first acrylic mold;
after the mixture being solidified in the third accommodating space to form a third block, magnetizing the third block, wherein the third block connects to another side of the first block, and the third block and the second block are disposed oppositely; and
taking out the first block, the second block, and the third block from the first acrylic mold to obtain the microrobot.

19. The method according to claim 18, wherein the weight ratio of the polydimethylsiloxane to the neodymium magnet particles of the second block is 1:4, and the weight ratio of the polydimethylsiloxane to the neodymium magnet particles of the third block is 1:4.

20. The method according to claim 18, wherein the microrobot has a length between 30 μm and 3000 μm, a width between 10 μm and 999 μm, and a height between 10 μm and 999 μm.
